(19) United States
(12) Patent Application Publication
(10) Pub. No.: US 2009/0240252 A1
(43) Pub. Date: Sep. 24, 2009

(54) TARGETING APPARATUS CONNECTING TO LOCKING NAILS FOR THE CORRECTION AND FIXATION OF FEMUR DEFORMITY OF A CHILD

(76) Inventor: Chia Hsieh Chang, Taichung City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/051,017

(22) Filed: Mar. 19, 2008

(51) Int. Cl. A61B 17/58

(52) U.S. Cl. 606/96

(57) ABSTRACT

A targeting apparatus for the correction of the deformity in proximal femur of a child is disclosed and includes a retention assembly; a cylindrical nail retention member tapered toward its half-spherical bottom end and including an upper through hole, a lower through hole, and a top cavity having inner threads for releasably secured to the retention assembly; a first locking nail including a forward threaded portion, an enlarged head, and a retaining recess in the head thereof; and a second locking nail including a forward threaded portion, an enlarged head, and a retaining recess in the head thereof. The invention has the advantages of reducing the area of wound when implanting the locking nails in the femur and saving labor during surgery.

### BACKGROUND OF THE INVENTION

1. Field of Invention

The invention relates to devices for correcting and fixing tubular bones, and more particularly to an improved targeting apparatus operatively connecting to locking nails for the correction of the deformity in proximal femur of a child.

2. Description of Related Art

Femoral varus osteotomy is one of the most common operations of children's hips to correct the dysplasia and deformity from neuromuscular disease, developmental dysplasia of the hip, Perthes' disease, trauma and infection. The surgical procedures include osteotomy at femoral intertrochanteric area, varus deformation of femoral neck, and internal fixation by implants. The universal implant in varus osteotomy is angled blade plate in which the femoral neck is fixed by one smooth blade. However, blade loosening from the cancelleous bone in femoral neck is not uncommon. The resulting loss of correction, malunion, nonunion, and prolonged immobilization are major surgical complications. Besides, a big wound and massive muscle dissection are required to implant the plate. Prominence at trochanteric area by the plate and the concomitant pain and discomfort are the common complains from the patients and parents.

A conventional targeting apparatus for the correction and fixation of femur deformation of a child is shown in FIG. 6. The targeting apparatus comprises an angled blade plate 50 having three tubular locating members (not numbered) for fastening three screws 51, and a thread-less blade 52 in which the screws 51 are threadedly inserted into the femoral shaft for fastening and the blade 52 is inserted into the femoral neck for fastening. However, the fastening of the blade 52 in the femoral neck is not reliable. Thus, time required for healing the femoral osteotomy may be prolonged undesirably. Moreover, the positioning of the angled blade plate 50 may cause a great pain to the leg. In addition, the angled blade plate 50 can cause bulging to the leg after surgery.

Therefore, the need for improvement still exists.

### SUMMARY OF THE INVENTION

It is therefore one object of the invention to provide a targeting apparatus operatively connecting to locking nails for the correction of the deformity in proximal femur of a child. The invention has the advantages as detailed below. The femoral shaft is fixed by three cortical bones and thus is more reliable as compared with the conventional angled blade plate. Wound on femur has a lesser area and cut muscle is less. Torque of the locking nails is relatively small and thus is better than the conventional angled blade plate. A doctor can use first and second locking nails to fix the femoral osteotomy in a labor saving manner. The leg only has a small bulging and no blade plate is disposed between the skin and the bone after surgery. The locking nails are inserted into positions below the femoral head and thus cause no injury to the body and do not hinder blood circulation.

To achieve the above and other objects, the invention provides a targeting apparatus for correcting the deformity in proximal femur of a child, comprising retention means; a cylindrical nail retention member tapered toward its half-spherical bottom end and including an upper through hole, a lower through hole, and a top cavity having inner threads for releasably secured to the retention means; a first locking nail including a forward threaded portion, an enlarged head, and a retaining recess in the head thereof; and a second locking nail including a forward threaded portion, an enlarged head, and a retaining recess in the head thereof.

The above and other objects, features and advantages of the invention will become apparent from the following detailed description taken with the accompanying drawings.

### BRIEF DESCRIPTION OF THE DRAWINGS

FIG. 1 is a perspective view of a preferred embodiment of targeting apparatus for the correction of the deformity in proximal femur of a child according to the invention;

FIG. 2 is an exploded view of FIG. 1 with the locking nail and the screw driver removed and a sleeve added;

FIG. 3 is an exploded view of FIG. 1;

FIG. 4 is a longitudinal sectional view of FIG. 3;

FIG. 5 schematically depicts the for the correction of the deformity in proximal femur in a child by means of the targeting apparatus operatively connected to the locking nails according to the invention; and

FIG. 6 schematically depicts a conventional targeting apparatus for the correction of the deformity in proximal femur of a child.

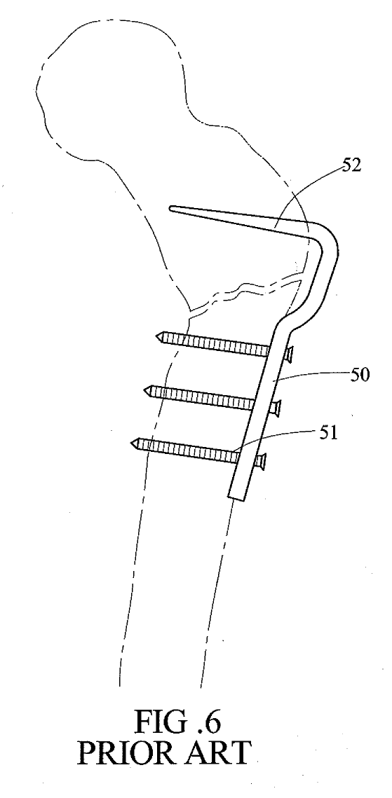

### DETAILED DESCRIPTION OF THE INVENTION

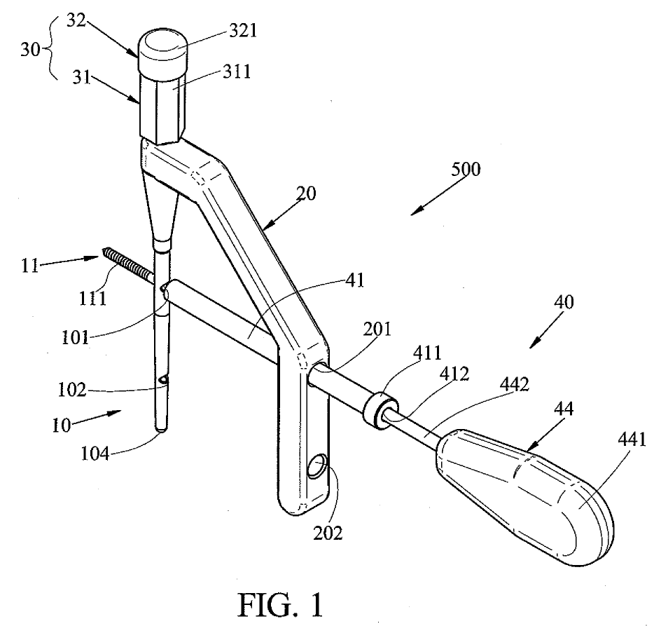

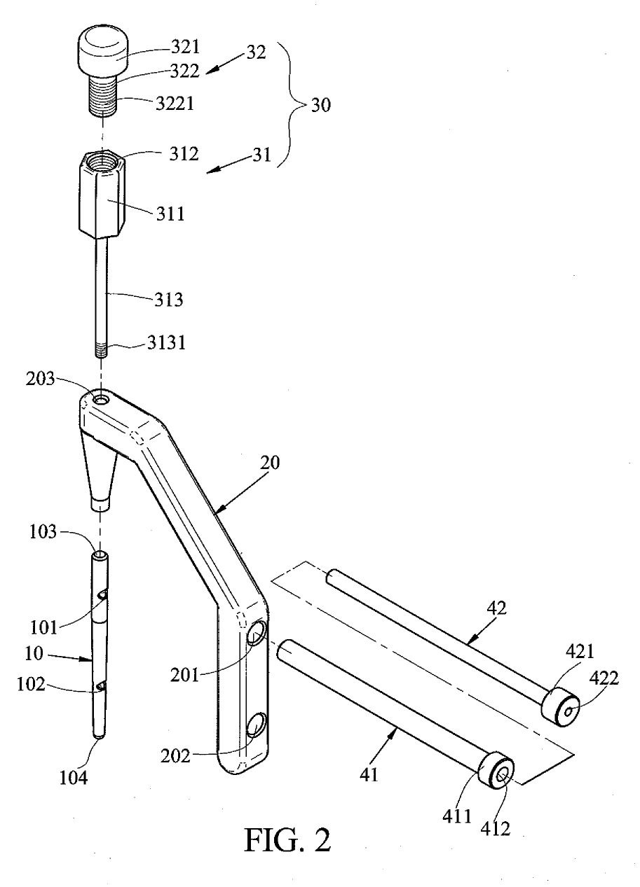

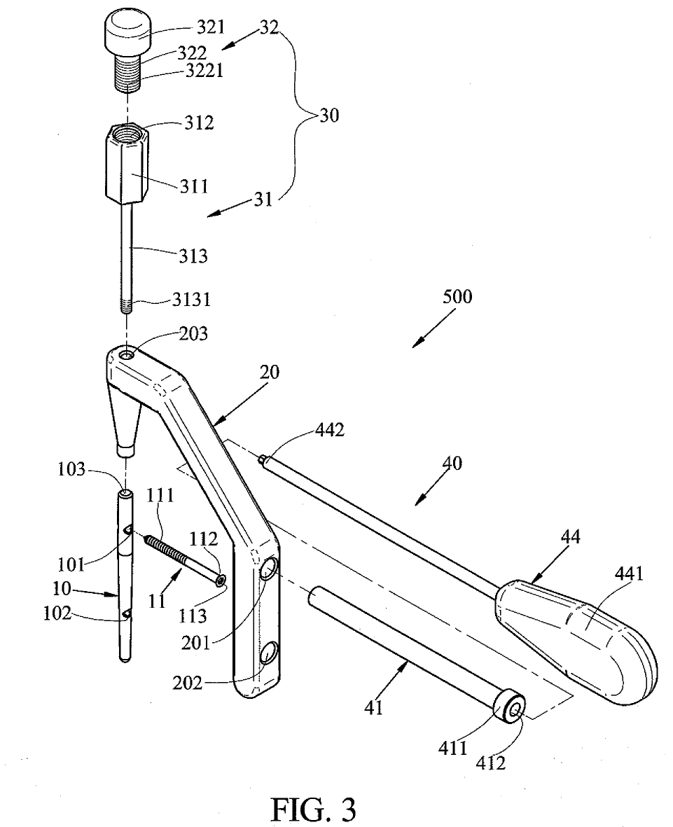

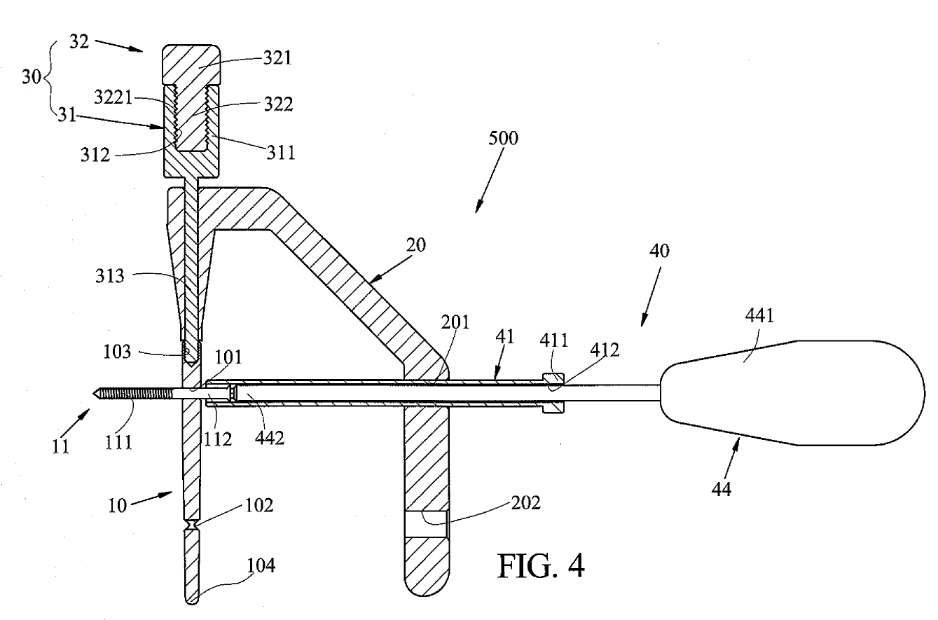

Referring to FIGS. 1 to 5, a targeting apparatus 500 for the correction of the deformity in proximal femur of a child in accordance with a preferred embodiment of the invention is shown. The targeting apparatus 500 operates in cooperation with a nail assembly (not numbered) which comprises the following components.

A nail retention member 10 is cylindrical. The nail retention member 10 is tapered toward its bottom end 104 which is half-spherical. The nail retention member 10 has an upper through hole 101, a lower through hole 102, and a top cavity 103 having inner threads.

A first locking nail 11 has a forward threaded portion 111, an enlarged head 112, and a recess 113 in the head 112.

A second locking nail 12 has a forward threaded portion 121, an enlarged head 122, and a recess (not numbered) in the head 122.

Preferably, the first locking nail 11 is a cannulated screw, and the second locking nail 12 is a cortical screw. Also, the first locking nail 11 has a diameter larger than that of the second locking nail 12.

The targeting apparatus 500 comprises the following components as detailed below. A handle 20 comprises an upright section having transverse upper and lower guide holes 201, 202, a horizontal section having a downward extension having a longitudinal channel 203 therethrough, and an inclined section interconnecting the upright and the horizontal sections.

A retention assembly 30 comprises a fastening unit 31 including a shank 313 having a threaded end 3131, a hexagonal head 311, and a recess 312 having inner threads in the head 311; and a retention unit 32 including an enlarged cylindrical head 321 and a shank 322 having outer threads 3221 which are adapted to threadedly secure to the recess 312.

A fastening assembly 40 comprises a joining sleeve 41 including an enlarged cylindrical head 411 and a central bore 412 through the joining sleeve 41; and a screw driver 44 including an enlarged handle 441 and a shank 442. The fastening assembly 40 further comprises a sleeve 42 including an enlarged cylindrical head 421, a shank (not numbered), and a central bore 422 through the shank. The shank of the sleeve 42 is adapted to insert into the central bore 412 of the joining sleeve 41.

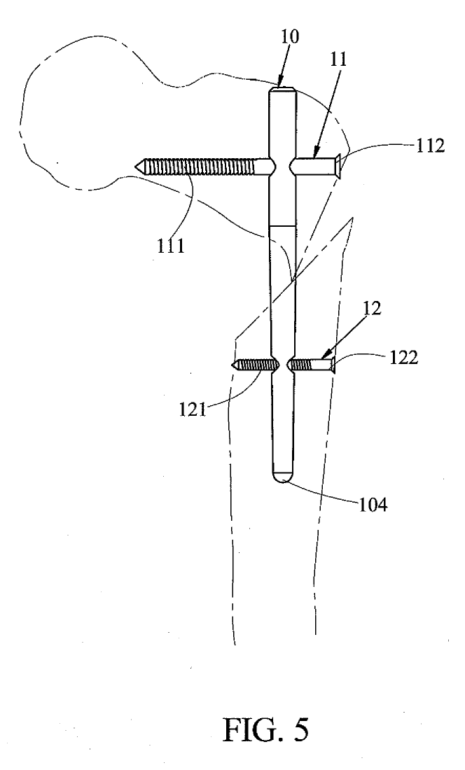

Referring to FIG. 5 in conjunction with FIGS. 1 to 4, an operation of the invention will be described in detail below. It is envisaged that the nail retention member 10 is adapted to insert into the femoral neck of a child in a neck-shaft angle. First, align the top cavity 103 with the channel 203. Next, threadedly secure the threaded end 3131 to the cavity 103 after inserting the threaded end 3131 through the channel 203 into the cavity 103. Next, use a hammer to pound the head 321 of the retention unit 32 until the nail retention member 10 is implanted in the femur. Next, cut the femur with the nail retention member 10 being fastened in the cortical bone. Next, insert the nail retention member 10 into the femur and turn the handle 20 to position the nail retention member 10. That is, the nail retention member 10 passes through the femoral neck and the femoral shaft. Next, use one hand to hold the handle and the other hand to insert the first locking nail 11 through the upper through hole 101. Next, insert the joining sleeve 41 through the upper guide hole 201 until the head 112 is received in the front portion of the joining sleeve 41. Next, insert the shank 442 of the screw driver 44 into the central bore 412 of the joining sleeve 41 until the end of the shank 442 engages the recess 113 in the head 112. Next, turn the screw driver 44 to drive the first locking nail 11 into the femoral neck. Similarly, use the other hand to insert the second locking nail 12 through the lower through hole 102. Next, insert the joining sleeve 41 through the lower guide hole 202 until the head 122 is received in the front portion of the joining sleeve 41. Next, insert the shank 442 of the screw driver 44 into the central bore 412 of the joining sleeve 41 until the end of the shank 442 engages the head 122. Next, turn the screw driver 44 to drive the second locking nail 12 into the femoral shaft. As a result, the femur is fixed to be readily for correction. Intramedullary interlocking nail fixation is the standard treatment for femoral fracture in adults. Intramedullary nailing bears less bending strain than plate fixation because the center of motion of nail is closer to the center of motion of femur. Minimal scaring of thigh muscles by intramedullary fixation allows early recovery of muscle function and bone healing. A new interlocking nail is specially designed for femoral varus osteotomy of a child. As the interlocking nail in adults, the nail is intramedullary fixation and offers additional stability by two locking screws. In addition, the nail is smaller in size (5 mm to 7 mm in diameter) to fit the smaller canal of a child's femurs. The nail is designed to insert form the area lateral to greater trochanter tip to avoid injury to structure and circulation of femoral head.

The nail system of the invention thus offers the following advantages as compared with the traditional blade plate. Better fixation at femoral neck by purchasing three locations of cortical bone. Biomechanical advantage of intramedullary fixation over plate fixation. Smaller wound and less muscle dissection for nail insertion. Less prominence at trochanter area after operation.

Moreover, the nail of the invention also offers the following technological advantages for the surgeons. It is possible of holding and targeting device for easier insertion of nail and locking screws. It is possible of controlling the anteversion of femur by two locking screws. Concomitant greater trochanter distal transfer is made possible.

While the invention herein disclosed has been described by means of specific embodiments, numerous modifications and variations could be made thereto by those skilled in the art without departing from the scope and spirit of the invention set forth in the claims.

What is claimed is:

1. A targeting apparatus for correcting the deformity in a proximal femur of a child, comprising:
retention means;
a cylindrical nail retention member tapered toward its half-spherical bottom end and including an upper through hole, a lower through hole, and a top cavity having inner threads for releasably secured to the retention means;
a first locking nail including a forward threaded portion, an enlarged head, and a retaining recess in the head thereof; and
a second locking nail including a forward threaded portion, an enlarged head, and a retaining recess in the head thereof.

2. The targeting apparatus of claim 1, wherein the first locking nail is a cannulated screw.

3. The targeting apparatus of claim 1, wherein the second locking nail is a cortical screw.

4. The targeting apparatus of claim 1, wherein the first locking nail has a diameter larger than that of the second locking nail.

5. The targeting apparatus of claim 1, wherein the retention means comprises:
a handle comprising an upright section having transverse upper and lower guide holes, a horizontal section having a downward extension having a longitudinal channel therethrough, and an inclined section interconnecting the upright and the horizontal sections wherein the cavity is adapted to align with the channel;
a retention assembly comprising a fastening unit including a shank having a threaded end, a head, and a recess having inner threads in the head thereof; and a retention unit including a head and a shank having outer threads adapted to threadedly secure to the recess of the fastening unit; and
a fastening assembly comprising a joining sleeve including a head and a central bore through the joining sleeve; and a screw driver including a handle and a shank,
wherein (i) the threaded end of the fastening unit is adapted to threadedly secure to the cavity after inserting through the channel into the cavity; (ii) the head of the retention unit is adapted to pound to implant the nail retention member in the femur; (iii) the femur is adapted to cut into a neck and a shaft; the nail retention member is adapted to position in the femoral neck and the femoral shaft by turning the handle; (iv) the first locking nail is adapted to insert through the upper through hole, the joining sleeve is adapted to insert through the upper guide hole until the head of the first locking nail is received in the front end of the joining sleeve, the shank of the screw driver is adapted to insert into the bore of the joining sleeve to engage the retaining recess of the first locking nail, and the screw driver is adapted to drive the first locking nail into the femoral neck; and (v) the second locking nail is adapted to insert through the lower through hole, the joining sleeve is adapted to insert through the lower guide hole until the head of the second locking nail is received in the front end of the joining sleeve, the shank of the screw driver is adapted to insert into the bore of the joining sleeve to engage the retaining recess of the second locking nail, and the screw driver is adapted to drive the second locking nail into the femoral shaft.

6. The targeting apparatus of claim 5, wherein the retention means further comprises a sleeving member including a head, a shank, and a central bore through the shank thereof, and wherein the shank of the sleeving member is adapted to insert into the bore of the joining sleeve.
